(19) United States
(12) Patent Application Publication
(10) Pub. No.: US 2025/0132012 A1
(43) Pub. Date: Apr. 24, 2025

(54) TREATMENT PLANNING SYSTEM, RADIOTHERAPY SYSTEM, AND COMPUTER-READABLE STORAGE MEDIUM

(71) Applicant: OUR UNITED CORPORATION, Xi'an (CN)

(72) Inventors: Hong CHENG, Xi'an (CN); Xikang ZHAO, Xi'an (CN)

(21) Appl. No.: 18/920,687

(22) Filed: Oct. 18, 2024

(30) Foreign Application Priority Data
Oct. 20, 2023 (CN) 202311369414.7
Oct. 20, 2023 (CN) 202311370306.1

(51) Int. Cl. G16H 20/40; A61N 5/10

(52) U.S. Cl. CPC G16H 20/40; A61N 5/1047; A61N 5/1081

(57) ABSTRACT

A treatment planning system is provided. The treatment planning system includes one or more processors, a memory, and one or more application programs, wherein the one or more application programs are stored in the memory and, when loaded and run by the one or more processors, cause the one or more processors to: acquire a target volume image; acquire arc information, wherein the arc information indicates that a rotation angle for which a radiation source continuously arcs along a first direction is greater than or equal to 360 degrees; acquire prescription dose information, wherein the prescription dose information includes target radiation doses for different regions; and generate a treatment plan based on the target volume image, the arc information, and the prescription dose information.

### CROSS-REFERENCE TO RELATED APPLICATION

This application is based on and claims priority to Chinese Patent Application No. 202311370306.1, filed on 20 Oct. 2023 and Chinese Patent Application No. 202311369414.7, filed on 20 Oct. 2023, the disclosures of which are herein incorporated by reference in their entireties.

### TECHNICAL FIELD

The present disclosure relates to the field of radiotherapy technologies, and in particular relates to a treatment planning system, a radiotherapy system, and a computer-readable storage medium.

### BACKGROUND

Radiotherapy is a common way to treat tumors, which is capable of killing tumor lesions using high-energy rays generated by a radiotherapy device.

### SUMMARY

Embodiments of the present disclosure provide a treatment planning system, a radiotherapy system, and a computer-readable storage medium.

The embodiments of the present disclosure provide a treatment planning system. The treatment planning system includes one or more processors, a memory, and one or more application programs, wherein the one or more application programs are stored in the memory and, when loaded and run by the one or more processors, cause the one or more processors to:
acquire a target volume image;
acquire arc information, wherein the arc information indicates that a rotation angle for which a radiation source continuously arcs along a first direction is greater than or equal to 360 degrees;
acquire prescription dose information, wherein the prescription dose information includes target radiation doses for different regions; and
generate a treatment plan based on the target volume image, the arc information, and the prescription dose information.

In some embodiments, the arc information indicates that the radiation source continuously arcs from a start point to an end point along the first direction; or
the arc information indicates that the radiation source arcs along the first direction and along a second direction, wherein the rotation angle for which the radiation source continuously arcs along the first direction is greater than or equal to 360 degrees.

In some embodiments, in the case that the arc information indicates that the radiation source arcs along the first direction and along the second direction, the arc information further indicates that a rotation angle for which the radiation source continuously arcs along the second direction is greater than or equal to 360 degrees.

In some embodiments, the one or more application programs, when loaded and run by the one or more processors, cause the one or more processors to:
acquire information of an arc mode, wherein the arc mode includes continuously arcing in a single direction or arcing forth and back.

In some embodiments, in a case that the arc information indicates that the radiation source continuously arcs in the single direction or indicates that a rotation angle for which the radiation source continuously arcs is greater than or equal to 360 degrees, the arc mode is continuously arcing in the single direction.

In some embodiments, acquiring the arc information includes:
acquiring start point information of the arcing and a rotation angle of the radiation source, wherein the rotation angle of the radiation source is greater than or equal to 360 degrees; or
acquiring around number of the arcing, start point information of each round of the arcing, and end point information of each round of the arc; or,
acquiring a total number of turns, start point information, and end point information of the arcing, wherein a rotation angle of the radiation source, determined based on the total number of turns, the start point information, and the end point information of the arcing, is greater than or equal to 360 degrees.

In some embodiments, the system further includes a display apparatus, and the one or more application programs, when loaded and run by the one or more processors, cause the one or more processors to:
control the display apparatus to display at least one of an arcing direction or a total number of turns of the arcing.

The embodiments of the present disclosure provide a radiotherapy system. The radiotherapy system includes a gantry capable of continuously rotating, a radiation source disposed on the gantry, and a control system, wherein
the gantry is configured to drive the radiation source to rotationally arc around a target object; and
the control system is configured to:
receive a treatment plan, wherein the treatment plan is generated based on a target volume image, arc information, and prescription dose information, the arc information indicating that a rotation angle for which the radiation source continuously arcs along a first direction is greater than or equal to 360 degrees;
control, based on the treatment plan, the radiation source to rotationally arc from a first start point along the first direction; and
control the radiation source to deliver a radiation beam to a target volume.

In some embodiments, the treatment plan includes a plurality of control points; wherein
the plurality of control points include the first start point and a first pause point, and the control system is configured to control the radiation source to continuously arc from the first start point to the first pause point along the first direction, wherein a rotation angle for which the radiation source continuously arcs from the first start point to the first pause point is greater than or equal to 360 degrees; or,
the plurality of control points include the first start point and an end point, and the control system is configured to control the radiation source to rotationally and unidirectionally arc from the first start point along the first direction until reaching the end point, wherein a rotation angle for which the radiation source continuously arcs from the first start point to the end point is greater than or equal to 360 degrees.

In some embodiments, the control system is further configured to control the radiation source to continuously arc along a second direction; and
the treatment plan includes a plurality of control points, the plurality of control points including the first start point, a first pause point, a second start point, and a second pause point, and the control system is configured to control the radiation source to continuously arc from the first start point to the first pause point along the first direction and continuously arc from the second start point to the second pause point along the second direction, wherein a rotation angle for which the radiation source continuously arcs from the first start point to the first pause point is greater than or equal to 360 degrees, and a rotation angle for which the radiation source continuously arcs from the second start point to the second pause point is greater than or equal to 360 degrees.

In some embodiments, the control system is further configured to control the radiation source to deliver, during a process of continuously and rotationally arcing, radiation to the target object.

In some embodiments, the radiotherapy system further includes a multi-leaf collimator configured for shaping a beam emitted by the radiation source, wherein the multi-leaf collimator includes two leaf groups opposite to each other, each leaf group including a plurality of leaves capable of moving independently; and
the control system is further configured to control any of the plurality of leaves of the multi-leaf collimator to continuously move at a time of the radiation source delivering radiation to the target object.

In some embodiments, the radiotherapy system further includes a multi-leaf collimator configured for shaping a beam emitted by the radiation source, wherein the multi-leaf collimator includes two leaf groups opposite to each other, each leaf group including a plurality of leaves capable of moving independently; and
the control system is further configured to:
control any of the plurality of leaves to move to any position within a leaf movement range during a continuously and rotationally arcing process of the radiation source.

The embodiments of the present disclosure provide a non-transitory computer-readable storage medium. The storage medium stores one or more computer programs, wherein the one or more computer programs, when loaded and executed by a processor of an electronic device, cause the electronic device to:
acquire a target volume image;
acquire arc information, wherein the arc information indicates that a rotation angle for which a radiation source continuously arcs along a first direction is greater than or equal to 360 degrees;
acquire prescription dose information, wherein the prescription dose information includes target radiation doses for different regions; and
generate a treatment plan based on the target volume image, the arc information, and the prescription dose information.

Details of one or more embodiments of the present disclosure are set forth in the following accompanying drawings and descriptions to make other features, objectives, and advantages of the present disclosure more concise and understandable.

### BRIEF DESCRIPTION OF DRAWINGS

The accompanying drawings described herein are used to help better understand the present disclosure and constitute a part of the present disclosure. The illustrative embodiments of the present disclosure and their descriptions are intended to explain the present disclosure and do not constitute undue limitations to the present disclosure. The drawings are as follows.

FIG. 1 is a schematic diagram of a radiotherapy system according to some embodiments of the present disclosure;

FIG. 2 is a schematic flowchart of the formulation of a treatment plan according to some embodiments of the present disclosure;

FIG. 3 is a schematic diagram of arc information according to some embodiments of the present disclosure;

FIG. 4(a) is a schematic diagram of another manner of arc information according to some embodiments of the present disclosure;

FIG. 4(b) is a schematic diagram of another manner of arc information according to some embodiments of the present disclosure;

FIG. 4(c) is a schematic diagram of another manner of arc information according to some embodiments of the present disclosure;

FIG. 5(a) is a schematic diagram of another manner of arc information according to some embodiments of the present disclosure;

FIG. 5(b) is a schematic diagram of another manner of arc information according to some embodiments of the present disclosure; and

FIG. 6 is a schematic flowchart of the formulation of a treatment plan according to some embodiments of the present disclosure.

### DETAILED DESCRIPTION

The technical solutions of embodiments of the present disclosure are described clearly and completely hereinafter in combination with the accompanying drawings in the embodiments of the present disclosure. It is obvious that the described embodiments are only a part of embodiments of the present disclosure, not all embodiments of the present disclosure. All the other embodiments acquired by those of ordinary skills in the art, based on the embodiments of the present disclosure without creative work, shall fall within the protection scope of the present disclosure.

It should be understood that the orientation or position relationships indicated by the terms “center,” “longitudinal,” “transversal,” “length,” “width,” “thickness,” “above,” “below,” “front,” “back,” “left,” “right,” “vertical,” “horizontal,” “top,” “bottom,” “inner,” “outer,” or the like are based on the orientation or position relationships as shown in the drawings, which are merely for ease of the description of the present disclosure and simplifying the description rather than indicating or implying that the indicated device or element must have a particular orientation or be constructed and operated in a particular orientation. Therefore, these terms should not be understood as a limitation to the present disclosure. In addition, the terms “first,” “second,” “third,” and the like are used for the purpose of description instead of indicating or implying relative importance or implicitly indicating the number of technical features indicated. Thus, the features defined by the terms “first,” “second,” “third,” and the like include cases of one or more of the features existing either explicitly or implicitly. In the descriptions of the present disclosure, unless otherwise stated specifically, the term “plurality” means two or more.

In the present disclosure, the word “exemplary” is configured to mean “serving as an example, illustration, or description.” Any embodiment described as “exemplary” in the present disclosure is not necessarily to be construed as preferred or advantageous over other embodiments. The following descriptions are presented to enable any person skilled in the art to implement and employ the present disclosure. In the following descriptions, details are set forth for the purpose of explanation. It is to be understood that those of ordinary skill in the art are able to realize that the present disclosure can be implemented without the use of these specific details. In other examples, well-known structures and procedures are not described in detail so as not to obscure the descriptions of the present disclosure with unnecessary details. Therefore, the present disclosure is not intended to be limited to the embodiments shown but to accord with the widest scope consistent with the principles and features disclosed in the present disclosure.

It should be noted that because the methods of the embodiments of the present disclosure are executed in a computer device, processing objects of each computer device exist in the form of data or information, such as time, which is essentially time information. It can be understood that in subsequent embodiments, when describing sizes, numbers, locations, etc., the corresponding data exists for processing by the computer device, which is not repeated in detail here.

The present disclosure relates to radiotherapy technologies. In some embodiments, radiation beams for radiotherapy include particle beams (such as neutron beams, proton beams, or electron beams), photon beams (such as X-rays or γ-rays), etc., or a combination thereof.

Usually, for performing radiotherapy on a patient's tumor, it is first necessary to formulate a radiotherapy plan based on the situation of the patient's tumor, and then a radiation device applies a desired radiation dose to the patient's tumor based on the treatment plan so as to achieve treatment for the patient's tumor.

Arc radiation is a type of radiotherapy. The are radiation is performed by making a radiation source rotationally arc around an isocenter of a radiation device, such that rays travel through healthy tissues along a non-fixed path, ensuring that the radiation dose to the healthy tissues is more dispersed to protect the healthy tissues and at the same time ensuring that a lesion at the isocenter receives the maximum radiation dose.

The present disclosure provides a treatment planning system, a radiotherapy system, and a computer-readable storage medium. With the cooperation of the treatment planning system and the radiotherapy system, arc radiation of greater than 360 degrees is achieved, which in turn shortens the radiotherapy time and improves the radiotherapy efficiency.

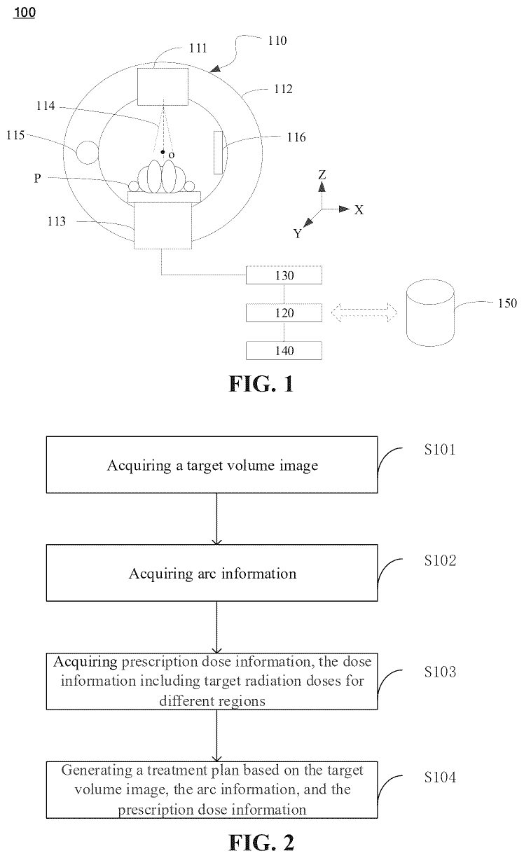

FIG. 1 exemplarily shows a radiotherapy system 100 according to some embodiments of the present disclosure. The radiotherapy system 100 includes a radiation delivery apparatus 110, a master control system 120, a slave control system 130, a treatment planning system (TPS) 140, and a memory 150. In some embodiments, the radiation delivery apparatus 110, the master control system 120, the slave control system 130, the treatment planning system 140, and the memory 150 are connected to and/or communicate with each other via wireless connection (e.g., network connection), wired connection, or a combination thereof.

In some embodiments, the radiation delivery apparatus 110 is an apparatus that delivers radiation therapy. In some embodiments, the radiation delivery apparatus 110 includes a radiation source 111, a rotating gantry 112, and a treatment couch 113.

The radiation source 111 is capable of generating or emitting a radiation beam 114. In some embodiments, the radiation source 111 includes a linear accelerator and a treatment head loaded with a radioisotope source (e.g., a cobalt-60 radiation source). A number of the radiation sources 111 is one or more. In some embodiments, there are two radiation sources 111.

The rotating gantry 112 is configured to support the radiation source 111 and is capable of driving the radiation source 111 to rotate around a rotation axis, and the rotation axis and a central axis of the radiation beam 114 intersect at an isocenter. In a radiation device provided by some embodiments of the present disclosure, the gantry is capable of continuously rotating for 360 degrees in a single direction. In some embodiments, the control and/or power supply connection of the gantry is achieved by a slip ring, such that the gantry is capable of continuously rotating for 360 degrees clockwise or counterclockwise.

The treatment couch 113 is configured to carry a patient P, and the treatment couch 113 is capable of translating in one or more of three orthogonal directions (shown as X, Y, and Z directions in FIG. 1). In some embodiments, the treatment couch 113 is further capable of rotating around any one or more of X, Y, and Z axes.

In some embodiments, the position of the radiation source 111 and the orientation of the radiation beam 114 relative to the patient are adjusted by controlling the movement of the rotating gantry 112 and/or the treatment couch 113.

In some embodiments, the radiation delivery apparatus 110 further includes an image guiding apparatus (including an imaging source 115 and a detector 116) configured to provide a medical image for determining at least a part (e.g., a region of interest) of the patient. In some embodiments, the image guiding apparatus includes a CT device, a cone-beam CT device, a PET device, a volume CT device, an MRI device, or the like, or a combination thereof.

In some embodiments, the master control system 120 is configured to generate a control instruction for one or more of the components (e.g., the slave control system 130, the treatment planning system 140, and the memory 150) of the radiotherapy system 100. In some embodiments, the master control system 120 sends an instruction to the slave control system 130 to control the radiation delivery apparatus 110 to start an image guidance or treatment process. In some embodiments, the master control system 120 sends an instruction to the treatment planning system 140 to acquire a treatment plan. In some embodiments, the instructions are input by a user (e.g., a doctor) via a user interface of the master control system 120.

In some embodiments, the slave control system 130 is configured to control the radiation delivery apparatus 110 to take a corresponding action in response to the control instruction generated by the master control system 120. In some embodiments, the slave control system 130 controls, based on a positioning instruction issued by the master control system 120, the movement of the treatment couch 113 of the radiation delivery apparatus 110 to achieve positioning. In some embodiments, the slave control system 130 controls, based on a radiation delivery instruction issued by the master control system 120, the movement of the rotating gantry 112 of the radiation delivery apparatus 110 to achieve radiation delivery. In some embodiments, the slave control system 130 controls, based on an image guidance instruction issued by the master control system 120, the image guiding apparatus of the radiation delivery apparatus 110 to perform image guidance on the patient and generate the medical image of the patient.

In some embodiments, the treatment planning system 140 is configured to determine the treatment plan based on a plan image of the patient (the plan image is an image of the patient acquired using an imaging apparatus prior to treatment) and/or based on at least a part of an object (e.g., a tumor) marked in the image acquired by the image guiding apparatus.

In some embodiments, both the master control system 120 and the treatment planning system 140 are computer devices with graphical user interfaces (GUI). The computer device includes one or more processors, a memory, and one or more application programs. In some embodiments, the one or more application programs in the treatment planning system 140 are stored in the memory and configured to, when loaded and executed by the one or more processors, cause the computer device to perform a method for generating a treatment plan described in the present disclosure. In some embodiments, the graphical user interface of the treatment planning system 140 is configured to interact with the user so as to formulate the treatment plan.

In some embodiments, the master control system 120 and the treatment planning system 140 are independent servers, or server networks or server clusters composed of multiple servers. In some embodiments, the computer device described in the embodiments of the present disclosure includes, but is not limited to, a computer, a network host, a single network server, a set of multiple network servers, or a cloud server composed of multiple servers. The cloud server is composed of a large number of computers or network servers based on cloud computing.

In some embodiments, the master control system 120 and the treatment planning system 140 are general-purpose computer devices or special-purpose computer devices. In specific implementations, the computer device is a desktop, a portable computer, a network server, a personal digital assistant (PDA), a mobile phone, a tablet computer, a wireless terminal device, a communication device, an embedded device, etc. The type of the computer device is not limited in the embodiments.

In some embodiments, the slave control system 130 is a computer device. The computer device includes a processor, a storage device, an input/output (I/O), and a communication port. In some embodiments, the processor 310 includes a microcontroller, a microprocessor, a reduced instruction set computer (RISC), an application-specific integrated circuit (ASIC), an application-specific instruction set processor (ASIP), a central processing unit (CPU), a graphic processing unit (GPU), a physical processing unit (PPU), a single chip microcomputer, a digital signal processor (DSP), a field-programmable gate array (FPGA), an advanced RISC machines (ARM) system, a programmable logic device (PLD), any circuit or processor capable of achieving one or more functions, etc., or any combination thereof.

In the radiotherapy system 100 according to the embodiments, when performing radiotherapy, the master control system 120 acquires the treatment plan configured for the treatment of the patient's tumor from the treatment planning system 140, and transmits the acquired treatment plan and the control instruction to the slave control system 130. The slave control system 130 controls the radiation delivery apparatus 110 to deliver radiotherapy to the patient's tumor based on treatment plan information and the control instruction.

In some embodiments, the radiotherapy system 100 further includes one or more other computer devices capable of processing data, such as an oncology information system (OIS). The OIS is configured to schedule the treatment plans of patients and store treatment data (such as image data of the patient, treatment plan data, and radiation delivery information.)

The memory 150 is configured to store data, one or more instructions, and/or any other information. In some embodiments, the memory 150 stores data acquired from the treatment planning system 140. In some embodiments, the memory 150 stores data and/or one or more instructions used by the master control system 120 to perform the illustrative method described in the present disclosure. In some embodiments, the memory 150 includes a mass memory, a removable memory, a volatile read-write memory, a read-only memory (ROM), etc., or any combination thereof. In some embodiments, the mass memory includes a magnetic disc, a compact disc, a solid-state drive, etc. In some embodiments, the removable memory includes a flash drive, a floppy disc, a compact disc, a memory card, a compression disc, a magnetic tape, etc. In some embodiments, the volatile read-write memory includes a random-access memory (RAM). In some embodiments, RAM includes a dynamic random-access memory (DRAM), a double data rate synchronous dynamic random-access memory (DDR SDRAM), a static random-access memory (SRAM), a thyristor random access memory (T-RAM), a zero-capacitance random-access memory (Z-RAM), etc. In some embodiments, ROM includes a mask ROM (MROM), a programmable ROM (PROM), an erasable programmable ROM (EPROM), an electrically erasable programmable ROM (EEPROM), a compact disc ROM (CD-ROM), a digital versatile disc ROM, etc. In some embodiments, the memory 150 is implemented on a cloud platform. Illustratively, the cloud platform includes a private cloud, a public cloud, a hybrid cloud, a community cloud, a distributed cloud, an internal cloud, a multi-tier cloud, etc., or any combination thereof.

In some embodiments, the memory 150 is connected to a network to communicate with one or more components (e.g., the master control system 120, the treatment planning system 140, or the OIS) of the radiotherapy system 100. The one or more components of the radiotherapy system 100 access data or one or more instructions stored in the memory 150 via the network. In some embodiments, the memory 150 is directly connected to or communicates with one or more components (e.g., the master control system 120, the treatment planning system 140, or the OIS) of the radiotherapy system 100. In some embodiments, the memory 150 is a part of the master control system 120, the treatment planning system 140, or the OIS.

It should be noted that the schematic diagram of a scene of the radiotherapy system shown in FIG. 1 is merely illustrative. The radiotherapy system and the scene described in the embodiments of the present disclosure are for the purpose of more clearly illustrating the technical solutions of the embodiments of the present disclosure, and do not constitute limitations to the technical solutions provided by the embodiments of the present disclosure. It is known by those of ordinary skill in the art that with the evolution of the radiotherapy system and the emergence of new business scenarios, the technical solutions provided by the embodiments of the present disclosure are still applicable to similar technical problems.

The present disclosure provides a treatment planning system. The treatment planning system includes one or more processors, a memory, and one or more application programs, wherein the one or more application programs are stored in the memory and, when loaded and run by the one or more processors, cause the one or more processors to perform the following processes as shown in FIG. 2.

In S101, a target volume image is acquired. In some embodiments, the target volume image is an image including a tumor or lesion, or the target volume image further includes an image of a tissue surrounding the tumor or lesion. In some embodiments, the target volume image is a three-dimensional image, including a CT, MR, or PET image, etc. In some embodiments, a series of images of the target volume are imported into the treatment planning system of the treatment device, and the treatment planning system fuses and registers different images, so as to accurately locate the patient's anatomical structure and determine the tumor's boundary, morphology, and distribution characteristics and the positions and shapes of healthy tissues with the help of the characteristics of different image forms, thereby facilitating accurately determining a target volume of treatment and a precise treatment protocol and avoiding sensitive organs.

In S102, arc information is acquired. The are information indicates that a rotation angle for which a radiation source continuously arcs along a first direction is greater than or equal to 360 degrees. In some embodiments, the first direction is clockwise or counterclockwise.

In some embodiments, the arc information indicating that the rotation angle for which the radiation source continuous arcs along the first direction is greater than or equal to 360 degrees includes one or more of the following cases. In a first case, the radiation source continuously arcs from a start point to an end point along the first direction, wherein the rotation angle for which the radiation source continuously arcs from the start point to the end point along the first direction is greater than or equal to 360 degrees. In a second case, the radiation source arcs along the first direction and along a second direction, wherein the rotation angle for which the radiation source continuously arcs along the first direction is greater than or equal to 360 degrees. In a third case, the radiation source rotationally arcs from the start point to a pause point along the first direction. and then rotationally arcs from the pause point along the second direction, wherein the rotation angle for which the radiation source continuously arcs from the start point to the pause point along the first direction is greater than or equal to 360 degrees, the second direction being different from the first direction. In a fourth case, the radiation source arcs along the first direction and along the second direction, wherein the rotation angle for which the radiation source continuously arcs along the first direction is greater than or equal to 360 degrees, and the rotation angle for which the radiation source continuously arcs along the second direction is greater than or equal to 360 degrees. In a fifth case, the radiation source rotationally arcs from the start point to the pause point along the first direction, and then rotationally arcs from the pause point along the second direction, wherein the rotation angle for which the radiation source arcs from the start point to the pause point along the first direction is greater than or equal to 360 degrees, and the rotation angle for which the radiation source continuously arcs from the pause point along the second direction is greater than or equal to 360 degrees.

In the following several embodiments, explanations are made by taking the example that the radiation source rotationally and continuously arcs from the start point along the first direction until reaching the end point, and the rotation angle of the radiation source is greater than or equal to 360 degrees. The first direction is clockwise or counterclockwise. It should be noted that in a conventional radiotherapy system, a large-volume power transmission cable is coupled to a rotating gantry, the cable is generally protected by a towing chain, and the angle that the gantry is capable of rotating is different according to the length of the towing chain. Therefore, protecting the cable using the towing chain makes it impossible for the rotating gantry to rotate continuously for more than 360 degrees. In the radiotherapy system according to the embodiments of the present disclosure, the control and/or power supply connection of the gantry is achieved by means of a slip ring, such that the gantry is capable of rotating continuously by 360 degrees clockwise or counterclockwise. That is, in the embodiments of the present disclosure, the rotation angle for which the radiation source continuously arcs from the start point to the end point along the first direction is greater than or equal to 360 degrees, which shortens the treatment duration and improves the treatment efficiency.

In some embodiments, the arc information includes a plurality of different parameters. The plurality of different parameters directly indicate that the rotation angle for which the radiation source continuously arcs from the start point to the end point along the first direction is greater than or equal to 360 degrees, or the rotation angle for which the radiation source continuously arcs from the start point to the end point along the first direction, that is calculated based on the plurality of different parameters, is greater than or equal to 360 degrees.

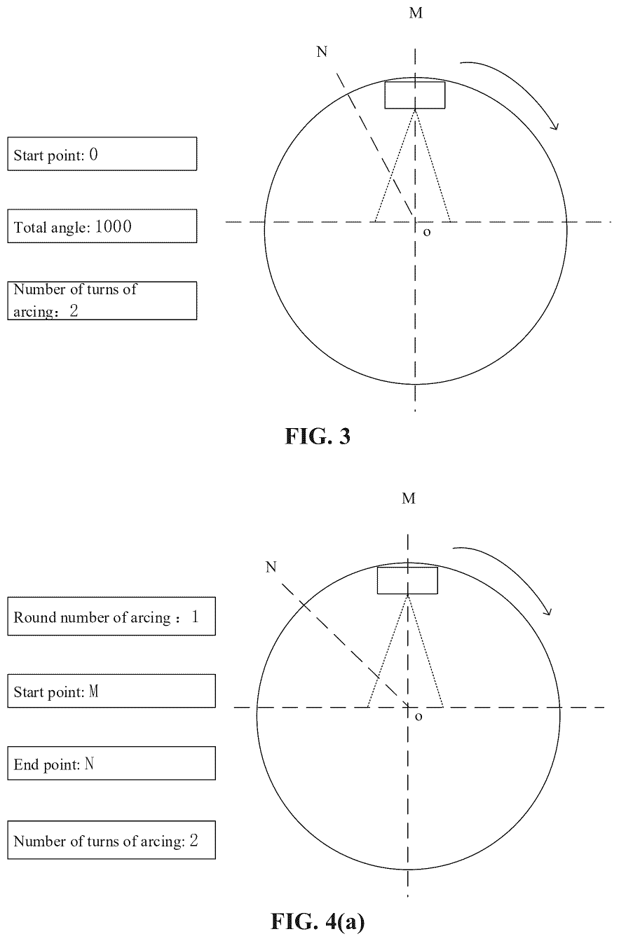

In some embodiments, acquiring the are information in S102 includes: acquiring start point information of the arcing; and acquiring a rotation angle of the radiation source, wherein the rotation angle of the radiation source is greater than or equal to 360 degrees. In some embodiments, the user inputs the corresponding start point or the rotation angle of the radiation source as required through the treatment planning system provided by the present disclosure, such that the treatment planning system acquires the arc information input by the user. In some embodiments, as shown in FIG. 3, by taking an example of an arcing direction as clockwise, the start point M at the 12 o'clock direction, and the rotation angle of the radiation source being 1,000 degrees, the treatment planning system calculates the position of the end point N based on the start point M and the rotation angle of the radiation source, and displays the end point N on a display apparatus. That is, the target arc information indicates that 2.78 turns of the arcing are completed from the start point. In other words, the radiation source completes 2 complete turns of rotationally arcing from the start point M, then completes 0.78 turns of rotationally arcing, and finally stops at the end point N. In some embodiments, the start point is at the 1 o'clock or 3 o'clock direction, which is not limited in the present disclosure. FIG. 3 only takes the start point M being at the 12 o'clock direction as an example for illustration.

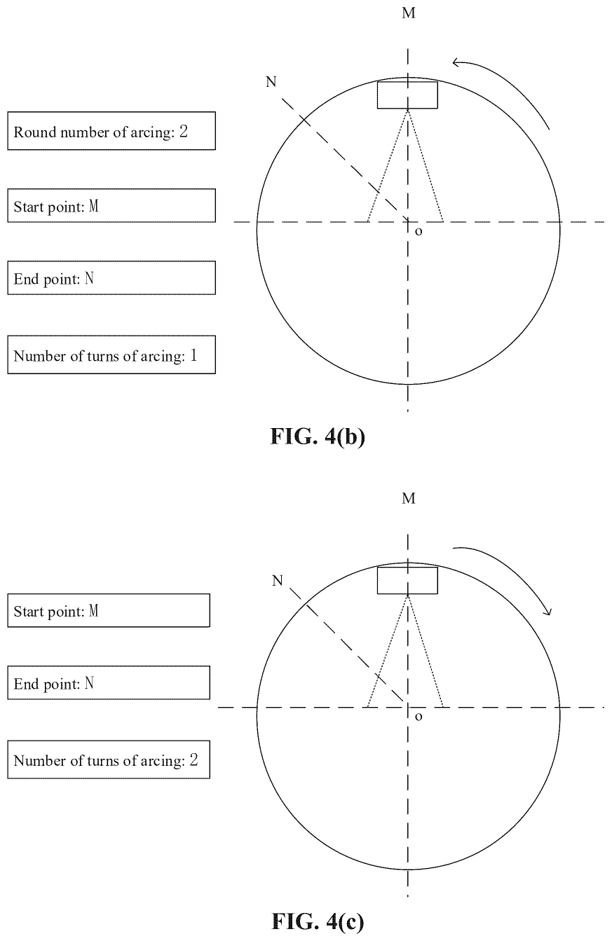

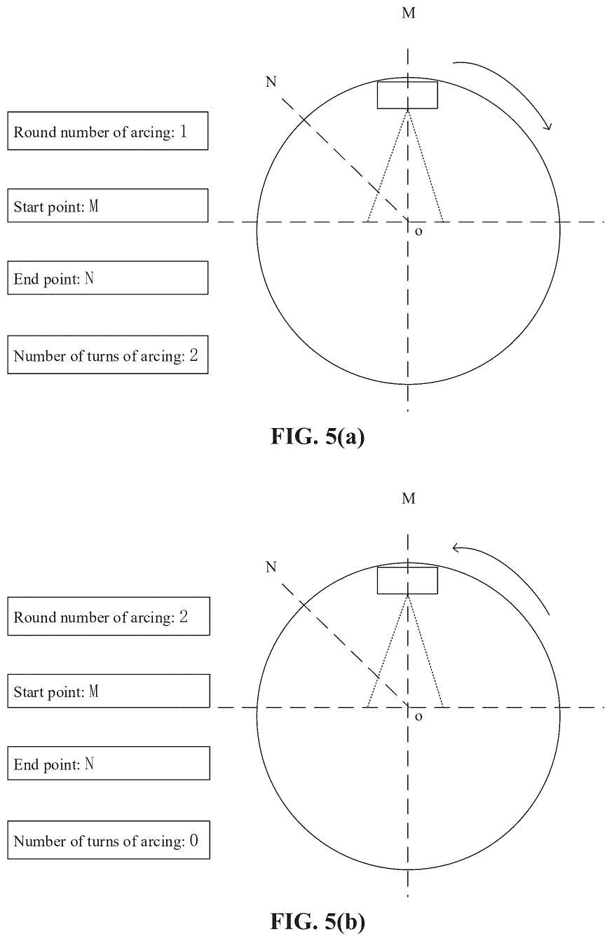

In some embodiments, acquiring the arc information in S102 includes: acquiring a round number of the arcing, start point information of each round of the arcing, and end point information of each round of the arcing. In some embodiments, through the treatment planning system according to the present disclosure, the user inputs the round number of the arcing, the start point information of each round of the arcing, and the end point information of each round of the arcing as required, such that the treatment planning system acquires the arc information input by the user. In some embodiments, as shown in FIG. 4(a), the arcing direction is clockwise, the round number of the arcing is 1 (i.e., the first round of arcing), the start point is M, the end point is N (seven-eighths of a turn), and the number of turns of the arcing is 2. That is, the radiation source needs to complete 2 complete turns of arcing and seven-eighths of a turn of arcing, with a rotation angle of 1,035 degrees. As shown in FIG. 4(b), the arcing direction is counterclockwise, the round number of the arcing is 2 (i.e., the second round of arcing), the start point is M, the end point is N (one-eighth of a turn), and the number of turns of the arcing is 1. That is, the radiation source needs to complete 1 complete turn of arcing and one-eighth of a turn of arcing, with a rotation angle of 405 degrees. Alternatively, as shown in FIG. 5(a), the arcing direction is clockwise, the round time of the arcing is 1 (i.e., the first round of arcing), the start point is M, the end point is N (seven-eighths of a turn), and the number of turns of the arcing is 2. That is, the radiation source needs to complete 2 complete turns of arcing and seven-eighths of a turn of arcing, with a rotation angle of 1,035 degrees. As shown in FIG. 5(b), the arcing direction is counterclockwise, the round number of the arcing is 2 (i.e., the second round of arcing), the start point is M, the end point is N (one-eighth of a turn), and the number of turns of the arcing is 0. That is, the radiation source needs to complete 0 and one-eighth of a turn of arcing, with a rotation angle of 45 degrees.

In some embodiments, acquiring the arc information in S102 includes: acquiring a total number of turns of the arcing; acquiring start point information of the arcing; and acquiring end point information of the arcing, wherein a rotation angle of the radiation source determined based on the total number of turns of the arcing, the start point information of the arcing, and the end point information of the arcing is greater than or equal to 360 degrees. In some embodiments, through the treatment planning system according to the present disclosure, the user inputs the total number of turns of the arcing, the start point of the arcing, and the end point of the arcing as required, such that the treatment planning system acquires the arc information input by the user. In some embodiments, as shown in FIG. 4(c), taking the arcing direction being clockwise as an example, the start point is M, the end point is N (seven-eighths of a turn), and the total number of turns of the arcing is 2. That is, the radiation source needs to complete 2 complete turns of arcing and seven-eighths of a turn of arcing, with a rotation angle of 1,035 degrees.

In some embodiments, the start point, the end point, and the round time of the arcing are set by the user as required, and the number of turns of the arcing is also set as required, which is not limited in the present disclosure. In some embodiments, the start point is at the 3 o'clock direction, the end point is at the 6 o'clock direction, and the round number of the arcing is 3. None of the start point, the end point, the number of turns of the arcing, and the round number of the arcing are limited in the present disclosure.

In some embodiments, the treatment planning system according to the present disclosure further includes a display apparatus, and the processor is further configured to control the display apparatus to display one or more of the arcing direction or the number of turns of the arcing. In the case where the radiation source rotationally and continuously arcs from the start point to the end point along the first direction, the processor is configured to control the display apparatus to display one or more of the direction of unidirectional arcing and the number of turns of the arcing. In some embodiments, as shown in FIGS. 3-5, the arcing direction is indicated by an arrow; and in some other embodiments, the arcing direction is indicated in other ways, such as text or dynamic graph. In some embodiments, the display apparatus further displays the number of turns of the arcing, as shown in FIG. 3, the number of turns of the arcing is shown as 2 turns. The display mode of neither the arcing direction nor the number of turns of the arcing is limited in the present disclosure, and is only illustrated by taking the above as an example.

In the following embodiments, exemplary explanations is made by taking that the radiation source arcs along the first direction and the second direction, wherein the rotation angle for which the radiation source continuously arcs along the first direction is greater than or equal to 360 degrees as an example.

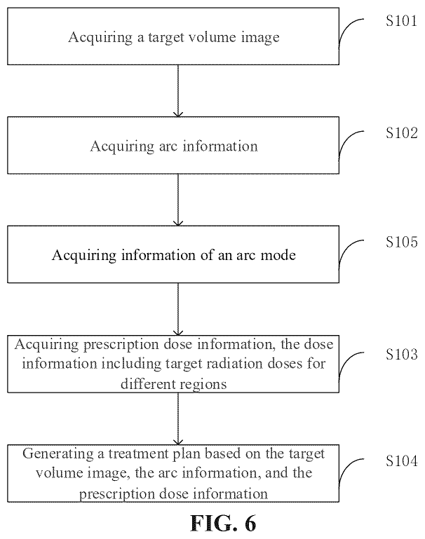

In some embodiments, as shown in FIG. 6, the processor of the treatment planning system provided by the embodiments of the present disclosure is further configured to perform process 105: acquiring information of an arc mode. It should be noted that there is no necessary sequence between process 105 and other processes. In some embodiments, either the information of the arc mode or the arc information is acquired first, which is not limited in the present disclosure and is only illustrated by taking FIG. 6 as an example. The arc mode includes continuously arcing in a single direction or arcing forth and back. In some embodiments, continuously arcing in a single direction includes continuously and multi-turn rotationally arcing clockwise or counterclockwise. In some embodiments, arcing forth and back includes rotating clockwise for a specific angle and then rotating counterclockwise. The radiotherapy system provided by the embodiments of the present disclosure is capable of continuously rotating for multiple turns. In some embodiments, the above-mentioned continuously arcing in a single direction means that the radiation source continuously arcs along the first direction from the start point to the end point, with the rotation angle of the radiation source being greater than or equal to 360 degrees. In some embodiments, the arcing forth and back means that the radiation source continuously arcs along the first direction from the start point to the pause point with the rotation angle of the radiation source being greater than or equal to 360 degrees, and then the radiation source continuously arcs along the second direction from the pause point with the rotation angle of the radiation source being greater than or equal to 360 degrees, which is not limited in the present disclosure.

In some embodiments, in the treatment planning system provided by the present disclosure, in the case that the arc information indicates that the radiation source continuously arcs in a single direction or the arc information indicates that the rotation angle of the radiation source is greater than or equal to 360 degrees, the arc mode is continuously arcing in a single direction. That is, in the case that the arc information indicates that the radiation source continuously arcs in a single direction, the arc mode is continuously arcing in a single direction. Alternatively, in the case that the arc information indicates that the rotation angle for which the radiation source continuously arcs is greater than or equal to 360 degrees, the arc mode is continuously arcing in a single direction. In this way, the radiation source preferentially unidirectionally and continuously arcs to avoid reverse rotation during the arc.

In some embodiments, the user is able to select the arc mode through the treatment planning system according to the present disclosure. Alternatively, in the case that the arc information indicates that the radiation source continuously arcs in a single direction or the arc information indicates that the rotation angle for which the radiation source continuously arcs is greater than or equal to 360 degrees, the treatment planning system automatically selects to continuously arc in a single direction.

In some embodiments, the first direction is clockwise, and the second direction is counterclockwise. In some embodiments, as shown in FIG. 5(a), the arcing direction is clockwise, the round number of the arcing is 1 (i.e., the first round of arcing), the start point is M, the end point is N (seven-eighths of a turn), and the number of turns of the arcing is 2. That is, the radiation source needs to complete 2 complete turns of arcing and seven-eighths of a turn of arcing, with a rotation angle of 1,035 degrees. As shown in FIG. 5(b), the arcing direction is counterclockwise, the round number of the arcing is 2 (i.e., the second round of the arcing), the start point is M, the end point is N (one-eighth of a turn), and the number of turns of the arcing is 0. That is, the radiation source needs to complete 0 and one-eighth of a turn of arcing, with a rotation angle of 45 degrees.

In the following several embodiments, exemplary explanations are made by taking that the radiation source arcs along the first direction and the second direction, with a total arc length for which the radiation source continuously arcs along the first direction being greater than an arc length of a predetermined radian, and a total arc length for which the radiation source continuously arcs along the second direction being greater than an arc length of a predetermined radian as an example, i.e., taking that the rotation angle for which the radiation source continuously arcs along the first direction is greater than a predetermined angle, and the rotation angle for which the radiation source continuously arcs along the second direction is greater than a predetermined angle as an example. In some embodiments, as shown in FIG. 4(a), the arcing direction is clockwise, the round number of the arcing is 1 (i.e., the first round of the arcing), the start point is M, the end point is N (seven-eighths of a turn), and the number of turns of the arcing is 2. That is, the radiation source needs to complete 2 complete turns of arcing and seven-eighths of a turn of arcing, with a rotation angle of 1,035 degrees. As shown in FIG. 4(b), the arcing direction is counterclockwise, the round number of the arcing is 2 (i.e., the second round of the arcing), the start point is M, the end point is N (one-eighth of a turn), and the number of turns of the arcing is 1. That is, the radiation source needs to complete 1 complete turn of arcing and one-eighth of a turn of arcing, with a rotation angle of 405 degrees.

As for the details of acquiring the arc information and how to implement the arcing, the present disclosure only illustrates the above embodiments by way of example. In fact, there are many different implementations, which are not limited in the present disclosure.

In S103, prescription dose information is acquired, wherein the prescription dose information includes target radiation doses for different regions. In some embodiments, the prescription dose information includes a prescription dose value, wherein the prescription dose value is a value of the dose received by a tumor treatment volume in the target treatment volume. In some embodiments, the value of the prescription dose varies according to the type and location of cancer cells, and the value of the corresponding dose is generally determined based on the specific characteristics of a tumor. In some embodiments, the value of the prescription dose is input by an attending physician, or the value of the prescription dose of the target treatment volume is selected based on the values of various parameters of the tumor and based on a pre-stored template.

In S104, a treatment plan is generated based on the target volume image, the arc information, and the prescription dose information.

In some embodiments, the treatment planning system generates a treatment plan based on the acquired target volume image, arc information, and prescription dose information. The radiotherapy system (device) receives the treatment plan and runs based on the treatment plan to achieve the purpose of radiotherapy. In some embodiments, the treatment plan includes a plurality of control points, wherein the plurality of control points include a start point and an end point; and the total arc length for which the radiation source continuously arcs from the start point to the end point along the first direction is greater than the arc length of a predetermined radian. That is, the radiation source rotationally and continuously arcs clockwise or counterclockwise from the start point until reaching the end point, wherein the rotation angle of the radiation source (2 turns as shown in FIG. 3 or FIG. 4) in this process is greater than or equal to 360 degrees.

In summary, the embodiments of the present disclosure provide a treatment planning system, including one or more processors, a memory, and one or more application programs, wherein the one or more application programs are stored in the memory and, when loaded and executed by the one or more processors, cause the one or more processors to: acquire a target volume image; acquire arc information, wherein the arc information indicates that a rotation angle for which a radiation source continuously arcs along a first direction is greater than or equal to 360 degrees; acquire prescription dose information, wherein the prescription dose information includes target radiation doses for different regions; and generate a treatment plan based on the target volume image, the arc information, and the prescription dose information. The arc information of the treatment plan indicates that the rotation angle for which the radiation source continuously arcs along the first direction is greater than or equal to 360 degrees. Therefore, for delivering radiation with an angle of greater than 360 degrees during arc radiation, the radiation source only rotationally and continuously arcs without reverse rotation, thereby improving the treatment efficiency.

Some embodiments of the present disclosure further provide a radiotherapy system. The radiotherapy system includes a gantry capable of continuously rotating, a radiation source disposed on the gantry, and a control system. The gantry is configured to drive the radiation source to rotationally arc around a target object (in some embodiments, the target object is a patient receiving radiotherapy, and the patient is on a treatment couch). The control system is configured to receive a treatment plan, wherein the treatment plan is generated based on a target volume image, arc information, and prescription dose information, the arc information indicating that a rotation angle for which the radiation source continuously arcs along a first direction is greater than or equal to 360 degrees. The control system is further configured to control, based on the treatment plan, the radiation source to rotationally arc along the first direction from a start point, wherein the rotation angle for which the radiation source rotationally arcs along the first direction is greater than or equal to 360 degrees. The control system is further configured to control the radiation source to emit a radiation beam to a target volume.

In some embodiments, the radiotherapy system 100 described above is as shown in FIG. 1. The radiotherapy system 100 includes a radiation delivery apparatus 110, a master control system 120, a slave control system 130, a treatment planning system (TPS) 140, and a memory 150. In some embodiments, the radiation delivery apparatus 110, the master control system 120, the slave control system 130. the treatment planning system 140, and the memory 150 are connected to and/or communicate with each other via wireless connection (such as, network connection), wired connection, or a combination thereof.

In the radiotherapy system provided by the embodiments of the present disclosure, the control system includes the master control system and the slave control system. In some embodiments, the master control system 120 is an upper computer and is capable of transmitting an instruction to the slave control system 130 (i.e., a lower computer), so as to control the radiation delivery apparatus 110 to perform treatment on a patient. In some embodiments, the master control system 120 transmits an instruction to the treatment planning system 140 to acquire a treatment plan.

The radiotherapy system acquires the treatment plan, and controls the movement of each component of the radiotherapy system based on the treatment plan, so as to accomplish the requirements of the treatment plan. In some embodiments, the treatment plan indicating that the rotation angle for which the radiation source continuously arcs along the first direction is greater than or equal to 360 degrees includes one or more of the following cases.

In some cases, the treatment plan includes a plurality of control points, the plurality control points including a first start point and an end point; and the control system is configured to control the radiation source to rotate unidirectionally from the first start point until reaching the end point, i.e., the radiation source continuously arcs in a single direction from the first start point to the end point, with a rotation angle being greater than or equal to 360 degrees. That is, for delivering radiation with an angle of more than 360 degrees during are radiation, the radiation source only needs to continuously and rotationally arc without reverse rotation, such that the treatment efficiency is improved.

In some other cases, the treatment plan includes a plurality of control points, the plurality of control points including a first start point and a first pause point; and the control system is configured to control the radiation source to continuously arc along the first direction from the first start point to the first pause point, wherein the rotation angle for which the radiation source continuously arcs from the first start point to the first pause point is greater than or equal to 360 degrees.

In some embodiments, the control system is further configured to control the radiation source to continuously arc along the second direction, wherein the rotation angle for which the radiation source continuously arcs along the second direction is greater than or equal to 360. In this case, the plurality of control points of the treatment plan include a first start point, a first pause point, a second start point, and a second pause point. The control system is configured to control the radiation source to continuously are from the first start point to the first pause point along the first direction and continuously arc from the second start point to the second pause point along the second direction, wherein the rotation angle for which the radiation source continuously arcs from the first start point to the first pause point is greater than or equal to 360 degrees, and the rotation angle for which the radiation source continuously arcs from the second start point to the second pause point is greater than or equal to 360 degrees. In some embodiments, in the case that the second pause point is an end point, or the round number of the arcing is more than 2, the rotation angle of the radiation source for each round of the arcing is greater than or equal to 360 degrees, or the rotation angle of the radiation source for part of the arcing is greater than or equal to 360 degrees, which is not limited in the present disclosure and only illustrated by taking the description and the accompanying drawings as an example.

In some embodiments, in the radiotherapy system provided by the present disclosure, the control system is further configured to control the radiation source to deliver radiation to a target object (in some embodiments, the target object being a patient) during a process of continuously and rotationally arcing.

In some embodiments, the control system controls, based on the requirements of the treatment plan, the radiation source to deliver radiation to the patient during the movement of continuous rotation of the radiation source around a longitudinal direction of the patient. In some embodiments, in the case that a radiation range of the radiation source specified in the treatment plan includes a plurality of radiation arc segments, the control system controls the radiation source to emit therapeutic rays to the patient in an arc segment that needs to be radiated by the radiation source, to achieve radiation delivery, and controls the radiation source to stop radiating in an arc segment that needs no radiation from the radiation source. In some embodiments, in the case that the radiation range of the radiation source specified in the treatment plan is one or more continuous complete turns (such as 1 turn, 2 turns, or N turns), the control system controls the radiation source to emit, during continuously rotating around the longitudinal direction of the patient, radiation rays to the patient, to achieve radiation delivery.

In some embodiments, the radiotherapy device of the radiotherapy system provided by the embodiments of the present disclosure further includes a multi-leaf collimator. In some embodiments, the multi-leaf collimator is configured for shaping the therapeutic beam emitted by the radiation source. In some embodiments of the present disclosure, the control system is further configured to control any of the plurality of leaves of the multi-leaf collimator to continuously move at a time of the radiation source delivering radiation to the target object (in some embodiments, the target object being a patient.)

The multi-leaf collimator includes two leaf groups opposite to each other, each leaf group includes a plurality of leaves arranged in the width directions of the leaves, and each of the plurality of leaves is capable of moving independently in a length direction of the corresponding leaf. Through the independent movements of the plurality of leaves, a hole adapted to the shape of the tumor is enclosed. Because the leaves of the multi-leaf collimator are made of materials (such as plumbum and tungsten) capable of shielding the therapeutic rays, the multi-leaf collimator is capable of shielding the therapeutic rays outside the hole, and therapeutic rays passing through the hole are delivered to a tumor region to achieve radiation delivery, so as to achieve beam-shaping of the therapeutic rays.

To make the tumor region receive a higher radiation dose and at the same time make the surrounding normal tissue receive the minimum radiation dose, the control system provided by the embodiments of the present disclosure is further configured to control any of the plurality of leaves of the multi-leaf collimator to continuously move at a time of the radiation source delivering radiation to the target object (in some embodiments, the target object is a patient). That is, during the multiple turns of continuously and rotationally arcing, the radiation source emits a beam and the leaves of the multi-leaf collimator move continuously, so as to achieve continuous multi-turn volumetric-modulate arc therapy (VMAT).

In some embodiments, the radiotherapy device of the radiotherapy system further includes a multi-leaf collimator for shaping the beam emitted by the radiation source. The multi-leaf collimator includes two leaf groups opposite to each other, and each leaf group includes a plurality of leaves capable of moving independently. The control system is further configured to control any of the plurality of leaves to move to any position within a leaf movement range during a continuously and rotationally arcing process of the radiation source. In some embodiments, the maximum movement range of the leaf of the multi-leaf collimator is 24 cm. In the present disclosure, the leaf is capable of stopping at any position within the maximum movement range (such as at position of 10 cm, 15 cm, 18 cm, or 20 cm; specifically, the leaf is controlled to stop according to the requirements of conformation and intensity modulation), such that the leaves are able to shield the radiation to achieve intensity modulation. In some embodiments, the maximum movement range of the multi-leaf collimator is 15 or 28 cm, etc., which is not specifically limited in the present disclosure and is only illustrated by taking the above as an example. In some embodiments, during the multi-turn continuously arcing process, the radiation source emits radiation, and at the same time, the leaves of the multi-leaf collimator move to achieve conformation and intensity modulation of beams, thereby achieving multi-turn volumetric-modulate arc therapy (VMAT).

To make the tumor region receive a higher radiation dose and at the same time make the surrounding normal tissue receive the minimum radiation dose, the control system provided by the embodiment of the present disclosure is further configured to control any of the plurality of leaves of the multi-leaf collimator to continuously move at a time of the radiation source delivering radiation to the target object (in some embodiments, the target object is the patient), i.e., the leaves of the multi-leaf collimator are also moving continuously at the time of the radiation source emitting a beam.

In some embodiments, the control system is further configured to control the treatment couch to remain stationary during the delivery of radiation from the radiation source to the target object (in some embodiments, the target object is a patient). That is, the control system controls the treatment couch to remain stationary (not moving) at the time of the radiation source emitting a beam.

In some embodiments, the control system provided by the embodiments of the present disclosure is further configured to control the treatment couch to move in a longitudinal direction of the patient during the delivery of radiation from the radiation source to the patient. Specifically, the control system controls the treatment couch to move unidirectionally or forth and back in the longitudinal direction (Y direction) of the patient at a uniform or non-uniform speed at the time of the radiation source emitting a beam.

In some embodiments, the radiotherapy device of the radiotherapy system provided by the embodiments of the present disclosure further includes an imaging apparatus, wherein the imaging apparatus is configured to capture an image of the patient; and the control system provided by the embodiments of the present disclosure is further configured to control the imaging apparatus to capture the image of the patient during the continuous rotational movement of the radiation source around the longitudinal direction of the patient.

As shown in FIG. 1, the imaging apparatus includes an imaging source 115 and a detector 116 arranged opposite to each other, wherein imaging rays emitted by the imaging source 115, upon passing through the tumor portion of the patient, are received by the detector 116, so as to generate an image of the tumor portion of the patient. In some embodiments, the control system controls the imaging apparatus to capture the image of the patient during the continuous rotational movement of the radiation source around the longitudinal direction of the patient. At the time of the imaging apparatus capturing the image of the patient, the radiation source is in a beam-emitting state or in a state of stopping emitting a beam, i.e., collection of the patient's image and beam emitting by the radiation source is carried out simultaneously or alternatively.

In some embodiments, the radiotherapy device of the radiotherapy system according to the embodiments of the present disclosure further includes a slip ring for achieving unlimited continuous rotation of the radiation source. The slip ring includes a stator and a rotor communicated through a conducting loop, wherein the transmission of power, signal or the like of the radiation source is achieved by means of the relative rotation between the stator to the rotor. In some embodiments, the slip ring has a stator lead-out wire connected to a power source/signal source, and a rotor lead-out wire connected to the radiation source. The rotational movement of the radiation source makes the stator and the rotor in rotary contact to provide power/a signal to the radiation source.

In some embodiments, a non-transitory computer-readable storage medium is provided. The storage medium stores one or more computer programs. The one or more computer programs, when loaded and run by a processor of an electronic device, cause the electronic device to: acquire a target volume image; acquire are information, wherein the arc information indicates that a rotation angle for which a radiation source continuously arcs along a first direction is greater than or equal to 360 degrees; acquire prescription dose information, wherein the prescription dose information includes target radiation doses for different regions; and generate a treatment plan based on the target volume image, the arc information, and the prescription dose information. The one or more computer programs in the computer-readable storage medium provided by the embodiments, when loaded and run by the processor of the electronic device, cause the electronic device to perform the method for generating a treatment plan according to the previous embodiments. A reference is made to the previous embodiments for details of the method, which is not repeated herein.

In some embodiments, the computer storage medium according to the embodiment of the present disclosure is implemented by any combination of one or more computer-readable mediums. The computer-readable medium is a computer-readable signal medium or a computer-readable storage medium. The computer-readable storage medium includes but is not limited to, an electrical, magnetic, optical, electromagnetic, infrared, or semiconductor system, apparatus, device, or any combination thereof. More specific examples (non-exhaustive list) of the computer-readable storage medium include a portable computer disk with an electrical connection by one or more wires, a hard disk, a random-access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination thereof. In the present disclosure, the computer-readable storage medium is any tangible medium that contains or stores one or more programs. The one or more programs are used by or used in combination with an instruction execution system, apparatus, or device.

In some embodiments, the computer-readable signal medium includes a data signal propagated in a baseband or as a part of a carrier wave, and one or more computer-readable program codes are carried in the data signal. This propagated data signal takes many forms, including but not limited to electromagnetic signals, optical signals, or any suitable combination thereof. In some embodiments, the computer-readable signal medium is any computer-readable medium other than the computer-readable storage medium. The computer-readable medium is capable of sending, propagating, or transmitting the one or more programs used by or used in combination with the instruction execution system, apparatus, or device.

The one or more program codes contained on the computer-readable medium are transmitted by any suitable medium, including but not limited to wireless, wire, optical cable, RF, etc., or any suitable combination thereof.

The one or more computer program codes used to perform the operations of the present disclosure are written in one or more programming languages or a combination thereof. The programming languages include object-oriented programming languages such as Java, Smalltalk, C++, and also include conventional procedural programming languages such as “C” language or similar programming languages. The one or more program codes are executed entirely on a user's computer, partly on the user's computer, executed as an independent software package, partly on the user's computer and partly executed on a remote computer, or entirely executed on the remote computer or server. In the case of a remote computer being involved, the remote computer is connected to the user's computer over any kind of network including a local area network (LAN) or a wide area network (WAN), or connected to an external computer (such as, over the Internet provided by an Internet service provider.)

It should be appreciated by those skilled in the art that in some embodiments, the above-described modules or processes of the present disclosure are achieved by a general-purpose computing device, the above-described modules or processes are centralized on a single computing device or distributed on a network formed by a plurality of computing devices. In some embodiments, the above-described modules or processes are implemented by program codes executable by a computing device, so that the above-described modules or processes are stored in a storage device to be executed by the computing device, or separately made into individual integrated circuit modules, or a plurality of the modules or processes are made into a single integrated circuit module. In this way, the present disclosure is not limited to any particular combination of hardware and software.

It is to be noted that the foregoing are only some embodiments of the present disclosure and the technical principles utilized by the present disclosure. It is understandable by those skilled in the art that the present disclosure is not limited to the particular embodiments described herein, and a variety of variations, readjustments, and substitutions apparent to those skilled in the art without departing from the protection scope of the present disclosure can be made. Therefore, although the present disclosure is described in detail by the above embodiments, the present disclosure is not limited to the above embodiments, but includes other equivalent embodiments without departing from the concept of the present disclosure, and the scope of the present disclosure is determined by the scope of the appended claims.

1. A treatment planning system, comprising one or more processors, a memory, and one or more application programs, wherein the one or more application programs are stored in the memory and, when loaded and run by the one or more processors, cause the one or more processors to:
acquire a target volume image;
acquire arc information, wherein the arc information indicates that a rotation angle for which a radiation source continuously arcs along a first direction is greater than or equal to 360 degrees;
acquire prescription dose information, wherein the prescription dose information comprises target radiation doses for different regions; and
generate a treatment plan based on the target volume image, the arc information, and the prescription dose information.

2. The treatment planning system according to claim 1, wherein the arc information indicates that the radiation source continuously arcs from a start point to an end point along the first direction; or
the arc information indicates that the radiation source arcs along the first direction and along a second direction, wherein the rotation angle for which the radiation source continuously arcs along the first direction is greater than or equal to 360 degrees.

3. The treatment planning system according to claim 2, wherein in the case that the arc information indicates that the radiation source arcs along the first direction and along the second direction, the arc information further indicates that a rotation angle for which the radiation source continuously arcs along the second direction is greater than or equal to 360 degrees.

4. The treatment planning system according to claim 1, wherein the one or more application programs, when loaded and run by the one or more processors, cause the one or more processors to:
acquire information of an arc mode, wherein the arc mode comprises continuously arcing in a single direction or arcing forth and back.

5. The treatment planning system according to claim 4, wherein in a case that the arc information indicates that the radiation source continuously arcs in the single direction or indicates that a rotation angle for which the radiation source continuously arcs is greater than or equal to 360 degrees, the arc mode is continuously arcing in the single direction.

6. The treatment planning system according to claim 1, wherein acquiring the arc information comprises:
acquiring start point information of the arcing and a rotation angle of the radiation source, wherein the rotation angle of the radiation source is greater than or equal to 360 degrees; or,
acquiring a round number of the arcing, start point information of each round of the arcing, and end point information of each round of the arc; or,
acquiring a total number of turns, start point information, and end point information of the arcing, wherein a rotation angle of the radiation source, determined based on the total number of turns, the start point information, and the end point information of the arcing, is greater than or equal to 360 degrees.

7. The treatment planning system according to claim 1, wherein the system further comprises a display apparatus, and the one or more application programs, when loaded and run by the one or more processors, cause the one or more processors to:
control the display apparatus to display at least one of an arcing direction or a total number of turns of the arcing.

8. A radiotherapy system, comprising a gantry capable of continuously rotating, a radiation source disposed on the gantry, and a control system, wherein
the gantry is configured to drive the radiation source to rotationally arc around a target object; and
the control system is configured to:
receive a treatment plan, wherein the treatment plan is generated based on a target volume image, arc information, and prescription dose information, the arc information indicating that a rotation angle for which the radiation source continuously arcs along a first direction is greater than or equal to 360 degrees;
control, based on the treatment plan, the radiation source to rotationally arc from a first start point along the first direction; and
control the radiation source to deliver a radiation beam to a target volume.

9. The radiotherapy system according to claim 8, wherein the treatment plan comprises a plurality of control points; wherein
the plurality of control points comprise the first start point and a first pause point, and the control system is configured to control the radiation source to continuously arc from the first start point to the first pause point along the first direction, wherein a rotation angle for which the radiation source continuously arcs from the first start point to the first pause point is greater than or equal to 360 degrees; or,
the plurality of control points comprise the first start point and an end point, and the control system is configured to control the radiation source to rotationally and unidirectionally arc from the first start point along the first direction until reaching the end point, wherein a rotation angle for which the radiation source continuously arcs from the first start point to the end point is greater than or equal to 360 degrees.

10. The radiotherapy system according to claim 8, wherein the control system is further configured to control the radiation source to continuously arc along a second direction; and
the treatment plan comprises a plurality of control points, the plurality of control points comprising the first start point, a first pause point, a second start point, and a second pause point, and the control system is configured to control the radiation source to continuously arc from the first start point to the first pause point along the first direction and continuously arc from the second start point to the second pause point along the second direction, wherein a rotation angle for which the radiation source continuously arcs from the first start point to the first pause point is greater than or equal to 360 degrees, and a rotation angle for which the radiation source continuously arcs from the second start point to the second pause point is greater than or equal to 360 degrees.

11. The radiotherapy system according to claim 8, wherein the control system is further configured to control the radiation source to deliver, during a process of continuously and rotationally arcing, radiation to the target object.

12. The radiotherapy system according to claim 8, wherein the radiotherapy system further comprises a multi-leaf collimator configured for shaping a beam emitted by the radiation source, wherein the multi-leaf collimator comprises two leaf groups opposite to each other, each leaf group comprising a plurality of leaves capable of moving independently; and
the control system is further configured to control any of the plurality of leaves of the multi-leaf collimator to continuously move at a time of the radiation source delivering radiation to the target object.

13. The radiotherapy system according to claim 8, wherein the radiotherapy system further comprises a multi-leaf collimator configured for shaping a beam emitted by the radiation source, wherein the multi-leaf collimator comprises two leaf groups opposite to each other, each leaf group comprising a plurality of leaves capable of moving independently; and
the control system is further configured to control any of the plurality of leaves to move to any position within a leaf movement range during a continuously and rotationally arcing process of the radiation source.

14. A non-transitory computer-readable storage medium, storing one or more computer programs, wherein the one or more computer programs, when loaded and executed by a processor of an electronic device, cause the electronic device to:
acquire a target volume image;
acquire arc information, wherein the arc information indicates that a rotation angle for which a radiation source continuously arcs along a first direction is greater than or equal to 360 degrees;
acquire prescription dose information, wherein the prescription dose information comprises target radiation doses for different regions; and
generate a treatment plan based on the target volume image, the arc information, and the prescription dose information.

15. The storage medium according to claim 14, wherein the arc information indicates that the radiation source continuously arcs from a start point to an end point along the first direction; or
the arc information indicates that the radiation source arcs along the first direction and along a second direction, wherein the rotation angle for which the radiation source continuously arcs along the first direction is greater than or equal to 360 degrees.

16. The storage medium according to claim 15, wherein in the case that the arc information indicates that the radiation source arcs along the first direction and along the second direction, the arc information further indicates that a rotation angle for which the radiation source continuously arcs along the second direction is greater than or equal to 360 degrees.

17. The storage medium according to claim 14, wherein the one or more computer programs, when loaded and executed by the processor of the electronic device, cause the electronic device to:
acquire information of an arc mode, wherein the arc mode comprises continuously arcing in a single direction or arcing forth and back.

18. The storage medium according to claim 17, wherein in a case that the arc information indicates that the radiation source continuously arcs in the single direction or indicates that a rotation angle for which the radiation source continuously arcs is greater than or equal to 360 degrees, the arc mode is continuously arcing in the single direction.

19. The storage medium according to claim 14, wherein the one or more computer programs, when loaded and executed by the processor of the electronic device, cause the electronic device to:
acquire start point information of the arcing and a rotation angle of the radiation source, wherein the rotation angle of the radiation source is greater than or equal to 360 degrees; or,
acquire a round number of the arcing, start point information of each round of the arcing, and end point information of each round of the arc; or,
acquire a total number of turns, start point information, and end point information of the arcing, wherein a rotation angle of the radiation source, determined based on the total number of turns, the start point information, and the end point information of the arcing, is greater than or equal to 360 degrees.

20. The storage medium according to claim 14, wherein the one or more computer programs, when loaded and executed by the processor of the electronic device, cause the electronic device to:
control a display apparatus to display at least one of an arcing direction or a total number of turns of the arcing.
